(19) United States
(12) Patent Application Publication
(10) Pub. No.: US 2002/0040136 A1
(43) Pub. Date: Apr. 4, 2002

(54) Vitronectin receptor antagonist

(75) Inventor: Dirk Heerding, Malvern, PA (US)

Correspondence Address: GLAXOSMITHKLINE, Corporate Intellectual Property - UW2220, P.O. Box 1539, King of Prussia, PA, 19406-0939, US

(73) Assignee: SmithKline Beecham Corporation

(21) Appl. No.: 09/996,141

(22) Filed: Nov. 28, 2001

Related U.S. Application Data
(63) Continuation of application No. 09/800,057, filed on Mar. 5, 2001.
Application No. 09/800,057 is a continuation of application No. 09/509,142, filed on Mar. 22, 2000, now abandoned.
Application No. 09/509,142 is a 371 of international application No. PCT/US98/19949, filed on Sep. 24, 1998.
(60) Provisional application No. 60/059,832, filed on Sep. 24, 1997.

(51) Int. Cl. A61K 31/55

(52) U.S. Cl. 540/523; 514/212.07

(57) ABSTRACT

A compound of the formula (I) is disclosed which is a vitronectin receptor antagonist and is useful in the treatment of osteoporosis:

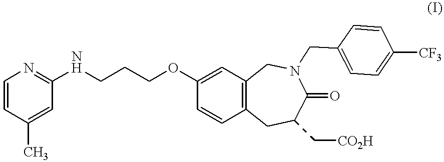

or a pharmaceutically acceptable salt thereof.

### FIELD OF THE INVENTION

This invention relates to a pharmaceutically active compound which inhibits the vitronectin receptor and is useful for the treatment of inflammation, cancer and cardiovascular disorders, such as atherosclerosis and restenosis, and diseases wherein bone resorption is a factor, such as osteoporosis.

### BACKGROUND OF THE INVENTION

Integrins are a superfamily of cell adhesion receptors, which are transmembrane glycoproteins expressed on a variety of cells. These cell surface adhesion receptors include gpIIb/IIIa (the fibrinogen receptor) and α_{V}β₃ (the vitronectin receptor). The fibrinogen receptor gpIIb/IIIa is expressed on the platelet surface, and mediates platelet aggregation and the formation of a hemostatic clot at the site of a bleeding wound. Philips, et al., Blood., 1988, 71, 831. The vitronectin receptor α_{V}β₃ is expressed on a number of cells, including endothelial, smooth muscle, osteoclast, and tumor cells, and, thus, it has a variety of functions. The α_{V}β₃ receptor expressed on the membrane of osteoclast cells mediates the adhesion of osteoclasts to the bone matrix, a key step in the bone resorption process. Ross, et al., J. Biol. Chem., 1987, 262, 7703. A disease characterized by excessive bone resorption is osteoporosis. The α_{V}β₃ receptor expressed on human aortic smooth muscle cells mediates their migration into neointima, a process which can lead to restenosis after percutaneous coronary angioplasty. Brown, et al., Cardiovascular Res., 1994, 28, 1815. Additionally, Brooks, et al., Cell, 1994, 79, 1157 has shown that an α_{V}β₃ antagonist is able to promote tumor regression by inducing apoptosis of angiogenic blood vessels. Thus, agents that block the vitronectin receptor would be useful in treating diseases, such as osteoporosis, restenosis and cancer.

The vitronectin receptor is now known to refer to three different integrins, designated α_{V}β₁, α_{V}β₃ and α_{V}β₅. Horton, et al., Int. J. Exp. Pathol., 1990, 71, 741. α_{V}β₁ binds fibronectin and vitronectin. α_{V}β₃ binds a large variety of ligands, including fibrin, fibrinogen, laminin, thrombospondin, vitronectin, von Willebrand's factor, osteopontin and bone sialoprotein I. α_{V}β₅ binds vitronectin. The vitronectin receptor α_{V}β₅ has been shown to be involved in cell adhesion of a variety of cell types, including microvascular endothelial cells, (Davis, et al., J. Cell. Biol., 1993, 51, 206), and its role in angiogenesis has been confirmed. Brooks, et al., Science, 1994, 264, 569. This integrin is expressed on blood vessels in human wound granulation tissue, but not in normal skin.

The vitronectin receptor is known to bind to bone matrix proteins which contain the tri-peptide Arg-Gly-Asp (or RGD) motif. Thus, Horton, et al., Exp. Cell Res. 1991, 195, 368, disclose that RGD-containing peptides and an anti-vitronectin receptor antibody (23C6) inhibit dentine resorption and cell spreading by osteoclasts. In addition, Sato, et al., J. Cell Biol. 1990, 111, 1713 discloses that echistatin, a snake venom peptide which contains the RGD sequence, is a potent inhibitor of bone resorption in tissue culture, and inhibits attachment of osteoclasts to bone.

It has now been discovered that a certain compound is a potent inhibitor of the α_{V}β₃ and α_{V}β₅ receptors. In particular, it has been discovered that such a compound is a more potent inhibitor of the vitronectin receptor than the fibrinogen receptor.

### SUMMARY OF THE INVENTION

This invention comprises a compound of the formula (I) as described hereinafter, which has pharmacological activity for the inhibition of the vitronection receptor and is useful in the treatment of inflammation, cancer and cardiovascular disorders, such as atherosclerosis and restenosis, and diseases wherein bone resorption is a factor, such as osteoporosis.

This invention is also a pharmaceutical composition comprising a compound according to formula (I) and a pharmaceutically carrier.

This invention is also a method of treating diseases which are mediated by the vitronectin receptor. In a particular aspect, the compound of this invention is useful for treating atherosclerosis, restenosis, inflammation, cancer and diseases wherein bone resorption is a factor, such as osteoporosis.

### DETAILED DESCRIPTION

This invention comprises a novel compound which is a more potent inhibitor of the vitronectin receptor than the fibrinogen receptor. The novel compound comprises a benzazepine core in which a nitrogen-containing substituent is present on the aromatic six-membered ring of the benzazepine and an aliphatic substituent containing an acidic moiety is present on the seven-membered ring of the benzazepine. The benzazepine ring system is believed to interact favorably with the vitronectin receptor and to orient the substituent sidechains on the six and seven membered rings so that they may also interact favorably with the receptor. It is preferred that about twelve to fourteen intervening covalent bonds via the shortest intramolecular path will exist between the acidic group on the aliphatic substituent of the seven-membered ring of the benzazepine and the nitrogen of the nitrogen-containing substituent on the aromatic six-membered ring of the benzazepine.

This invention comprises a compound of formula (I):

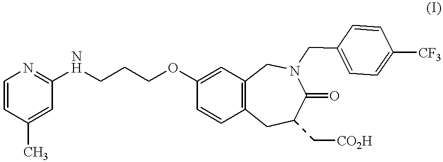

or a pharmaceutically acceptable salt thereof. This compound is (S)-8-[3-(4-methylpyridin-2-ylamino)-1-propyloxy]-3-oxo-2-[4-(trifluoromethyl)benzyl]-2, 3, 4, 5-tetrahydro-1H-2-benzazepine-4acetic acid.

The compound of formula (I) inhibits the binding of vitronectin and other RGD-containing peptides to the vitronectin receptor. Inhibition of the vitronectin receptor on osteoclasts inhibits osteoclastic bone resorption and is useful in the treatment of diseases wherein bone resorption is associated with pathology, such as osteoporosis and osteoarthritis.

In another aspect, this invention is a method for stimulating bone formation which comprises administering a compound of formula (I) which causes an increase in osteocalcin release. Increased bone production is a clear benefit in disease states wherein there is a deficiency of mineralized bone mass or remodeling of bone is desired, such as fracture healing and the prevention of bone fractures. Diseases and metabolic disorders which result in loss of bone structure would also benefit from such treatment. For instance, hyperparathyroidism, Paget's disease, hypercalcemia of malignancy, osteolytic lesions produced by bone metastasis, bone loss due to immobilization or sex hormone deficiency, Behcet's disease, osteomalacia, hyperostosis and osteopetrosis, could benefit from administering a compound of this invention.

Additionally, since the compound of the instant invention inhibits vitronectin receptors on a number of different types of cells, said compound would be useful in the treatment of inflammatory disorders, such as rheumatoid arthritis and psoriasis, and cardiovascular diseases, such as atherosclerosis and restenosis. The compound of formula (I) of the present invention may be useful for the treatment or prevention of other diseases including, but not limited to, thromboembolic disorders, asthma, allergies, adult respiratory distress syndrome, graft versus host disease, organ transplant rejection, septic shock, eczema, contact dermatitis, inflammatory bowel disease, and other autoimmune diseases. The compound of the present invention may also be useful for wound healing.

The compound of the present invention is also useful for the treatment, including prevention, of angiogenic disorders. The term angiogenic disorders as used herein includes conditions involving abnormal neovascularization. Where the growth of new blood vessels is the cause of, or contributes to, the pathology associated with a disease, inhibition of angiogenisis will reduce the deleterious effects of the disease. An example of such a disease target is diabetic retinopathy. Where the growth of new blood vessels is required to support growth of a deleterious tissue, inhibition of angiogenisis will reduce the blood supply to the tissue and thereby contribute to reduction in tissue mass based on blood supply requirements. Examples include growth of tumors where neovascularization is a continual requirement in order that the tumor grow and the establishment of solid tumor metastases. Thus, the compound of the present invention inhibit tumor tissue angiogenesis, thereby preventing tumor metastasis and tumor growth.

Thus, according to the methods of the present invention, the inhibition of angiogenesis using the compound of the present invention can ameliorate the symptoms of the disease, and, in some cases, can cure the disease.

Another therapeutic target for the compound of the instant invention are eye diseases characterized by neovascularization. Such eye diseases include corneal neovascular disorders, such as corneal transplantation, herpetic keratitis, luetic keratitis, pterygium and neovascular pannus associated with contact lens use. Additional eye diseases also include age-related macular degeneration, presumed ocular histoplasmosis, retinopathy of prematurity and neovascular glaucoma.

This invention further provides a method of inhibiting tumor growth which comprises administering stepwise or in physical combination a compound of formula (I) and an antineoplastic agent, such as topotecan and cisplatin.

Also included in this invention are prodrugs of the compounds of this invention. Prodrugs are considered to be any covalently bonded carriers which release the active parent drug according to formula (I) in vivo. Thus, in another aspect of this invention are novel prodrugs, which are also intermediates in the preparation of the formula (I) compound, of formula (II):

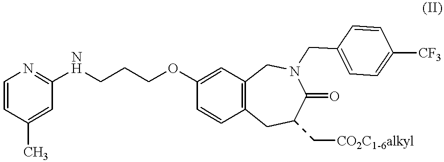

or a pharmaceutically acceptable salt thereof.

In yet another aspect of this invention are novel intermediates of formula (III):

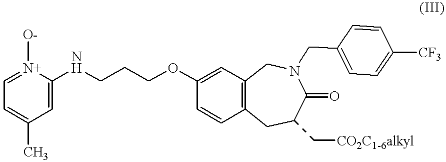

or a pharmaceutically acceptable salt thereof.

Abbreviations and symbols commonly used in the peptide and chemical arts are used herein to describe the compounds of this invention. In general, the amino acid abbreviations follow the IUPAC-IUB Joint Commission on Biochemical Nomenclature as described in Eur. J. Biochem., 158, 9 (1984).

C₁₋₆alkyl as applied herein means an optionally substituted alkyl group of 1 to 6 carbon atoms, and includes methyl, ethyl, n-propyl, isopropyl, n-butyl, isobutyl, t-butyl, n-pentyl, isopentyl, neopentyl and hexyl and the simple aliphatic isomers thereof.

Certain reagents are abbreviated herein. DCC refers to dicyclohexylcarbodiimide, DMAP refers to dimethylaminopyridine, DIEA refers to diisopropylethyl amine, EDC refers to 1-(3-dimethylaminopropyl)-3-ethylcarbodiimide, hydrochloride. HOBt refers to 1-hydroxybenzotriazole, THF refers to tetrahydrofuran, DIEA refers to diisopropylethylamine, DEAD refers to diethyl azodicarboxylate, PPh₃ refers to triphenylphosphine, DIAD refers to diisopropyl azodicarboxylate, DME refers to dimethoxyethane, DMF refers to dimethylformamide, NBS refers to N-bromosuccinimide, Pd/C refers to a palladium on carbon catalyst, PPA refers to polyphosphoric acid, DPPA refers to diphenylphosphoryl azide, BOP refers to benzotriazol-1-yloxy-tris(dimethylamino)phosphonium hexafluorophosphate, HF refers to hydrofluoric acid, TEA refers to triethylamine, TFA refers to trifluoroacetic acid, PCC refers to pyridinium chlorochromate.

Compounds of the formula (I) are generally prepared by the methods described in Bondinell, et al., PCT application WO 93/00095, published Jan. 7, 1993 and Bondinell, et al., PCT application WO 94/14776, the entire disclosures of which are incorporated herein by reference.

Additionally, the compound of formula (I) is prepared by the methods detailed in the scheme hereinbelow.

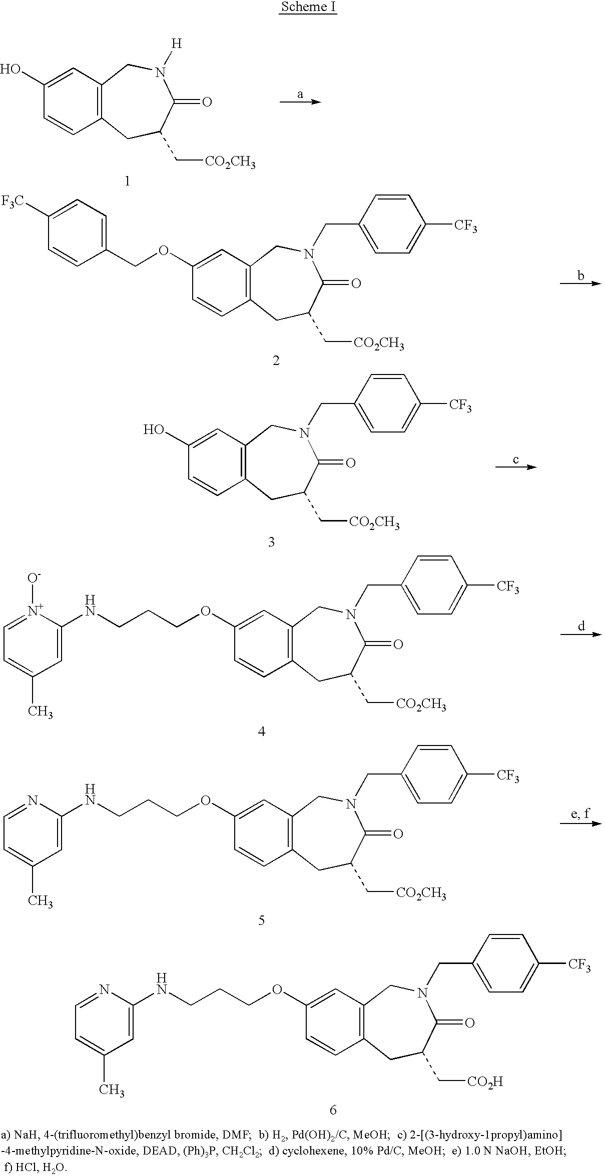

Compound I-1, prepared by the general procedures described in Bondinell, et al., PCT application WO 93/00095, published Jan. 7, 1993 and Bondinell, et al., PCT application WO 94/14776, is reacted with 4-(trifluoromethyl)benzyl bromide in the presence of a suitable base, generally sodium hydride or lithium bis(trimethylsilyl)amide, in an aprotic solvent, preferably DMF, THF, or mixtures thereof, to afford the bis-alkylated product I-2. The 4-(trifluoromethyl)benzyl ether of I-2 can be conveniently removed by hydrogenolysis to provide the phenol I-3. Methods for hydrogenolysis of benzyl ethers are well-known to those of skill in the art, and are described in appropriate reference volumes, for instance in Greene, “Protective Groups in Organic Synthesis” (published by Wiley-Interscience). Compound I-3, is reacted with 2-[(3-hydroxy-1-propyl)amino]-4-methylpyridine-N-oxide in a Mitsunobu-type coupling reaction (Organic Reactions 1992, 42, 335-656; Synthesis 1981, 1-28) to afford I-4. The reaction is mediated by the complex formed between diethyl azodicarboxylate and triphenylphosphine, and is conducted in an aprotic solvent, for instance THF, CH₂Cl₂, or DMF. The pyridine-N-oxide moiety of I-4 is reduced to the corresponding pyridine I-5 under transfer hydrogenation conditions using a palladium catalyst, preferably palladium metal on activated carbon, in an inert solvent, for instance methanol, ethanol, or 2-propanol. Cyclohexene, 1,4-cyclohexadiene, formic acid, and salts of formic acid, such as potassium formate or ammonium formate, are commonly used as the hydrogen transfer reagent in this type of reaction. The methyl ester of I-5 is hydrolyzed using aqueous base, for example, LiOH in aqueous THF or NaOH in aqueous methanol or ethanol, and the intermediate carboxylate salt is acidified with a suitable acid, for instance TFA or HCl, to afford the carboxylic acid I-6. Alternatively, the intermediate carboxylate salt can be isolated, if desired, or a carboxylate salt of the free carboxylic acid can be prepared by methods well-known to those of skill in the art.

Acid addition salts of the compound are prepared in a standard manner in a suitable solvent from the parent compound and an excess of an acid, such as hydrochloric, hydrobromic, hydrofluoric, sulfuric, phosphoric, acetic, trifluoroacetic, maleic, succinic or methanesulfonic. Cationic salts are prepared by treating the parent compound with an excess of an alkaline reagent, such as a hydroxide, carbonate or alkoxide, containing the appropriate cation; or with an appropriate organic amine. Cations such as Li⁺, Na⁺, K⁺, Ca⁺⁺, Mg⁺⁺ and NH₄⁺ are specific examples of cations present in pharmaceutically acceptable salts.

This invention also provides a pharmaceutical composition which comprises a compound according to formula (I) and a pharmaceutically acceptable carrier. Accordingly, the compound of formula (I) may be used in the manufacture of a medicament. Pharmaceutical compositions of the compound of formula (I) prepared as hereinbefore described may be formulated as solutions or lyophilized powders for parenteral administration. Powders may be reconstituted by addition of a suitable diluent or other pharmaceutically acceptable carrier prior to use. The liquid formulation may be a buffered, isotonic, aqueous solution. Examples of suitable diluents are normal isotonic saline solution, standard 5% dextrose in water or buffered sodium or ammonium acetate solution. Such formulation is especially suitable for parenteral administration, but may also be used for oral administration or contained in a metered dose inhaler or nebulizer for insufflation. It may be desirable to add excipients such as polyvinylpyrrolidone, gelatin, hydroxy cellulose, acacia, polyethylene glycol, mannitol, sodium chloride or sodium citrate.

Alternately, the compound may be encapsulated, tableted or prepared in a emulsion or syrup for oral administration. Pharmaceutically acceptable solid or liquid carriers may be added to enhance or stabilize the composition, or to facilitate preparation of the composition. Solid carriers include starch, lactose, calcium sulfate dihydrate, terra alba, magnesium stearate or stearic acid, talc, pectin, acacia, agar or gelatin. Liquid carriers include syrup, peanut oil, olive oil, saline and water. The carrier may also include a sustained release material such as glyceryl monostearate or glyceryl distearate, alone or with a wax. The amount of solid carrier varies but, preferably, will be between about 20 mg to about 1 g per dosage unit. The pharmaceutical preparations are made following the conventional techniques of pharmacy involving milling, mixing, granulating, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

For rectal administration, the compound of this invention may also be combined with excipients such as cocoa butter, glycerin, gelatin or polyethylene glycols and molded into a suppository.

The compound described herein is an antagonist of the vitronectin receptor, and is useful for treating diseases wherein the underlying pathology is attributable to ligand or cell which interacts with the vitronectin receptor. For instance, this compound is useful for the treatment of diseases wherein loss of the bone matrix creates pathology. Thus, the instant compound is useful for the treatment of ostoeporosis, hyperparathyroidism, Paget's disease, hypercalcemia of malignancy, osteolytic lesions produced by bone metastasis, bone loss due to immobilization or sex hormone deficiency. The compound of this invention is also believed to have utility as an antitumor, anti-angiogenic, antiinflammatory and anti-metastatic agent, and be useful in the treatment of atherosclerosis and restenosis.

The compound is administered either orally or parenterally to the patient, in a manner such that the concentration of drug is sufficient to inhibit bone resorption, or other such indication. The pharmaceutical composition containing the compound is administered at an oral dose of between about 0.1 to about 50 mg/kg in a manner consistent with the condition of the patient. Preferably the oral dose would be about 0.5 to about 20 mg/kg. For acute therapy, parenteral administration is preferred. An intravenous infusion of the peptide in 5% dextrose in water or normal saline, or a similar formulation with suitable excipients, is most effective, although an intramuscular bolus injection is also useful. Typically, the parenteral dose will be about 0.01 to about 100 mg/kg; preferably between 0.1 and 20 mg/kg. The compound is administered one to four times daily at a level to achieve a total daily dose of about 0.4 to about 400 mg/kg/day. The precise level and method by which the compound is administered is readily determined by one routinely skilled in the art by comparing the blood level of the agent to the concentration required to have a therapeutic effect.

This invention further provides a method for treating osteoporosis or inhibiting bone loss which comprises administering stepwise or in physical combination a compound of formula (I) and other inhibitors of bone resorption, such as bisphosphonates (i.e., allendronate), hormone replacement therapy, anti-estrogens, or calcitonin. In addition, this invention provides a method of treatment using a compound of this invention and an anabolic agent, such as the bone morphogenic protein, iproflavone, useful in the prevention of bone loss and/or to increase bone mass.

Additionally, this invention provides a method of inhibiting tumor growth which comprises administering stepwise or in physical combination a compound of formula (I) and an antineoplastic agent. Compounds of the camptothecin analog class, such as topotecan, irinotecan and 9-aminocamptothecin, and platinum coordination complexes, such as cisplatin, ormaplatin and tetraplatin, are well known groups of antineoplastic agents. Compounds of the camptothecin analog class are described in U.S. Pat. Nos. 5,004,758, 4,604,463, 4,473,692, 4,545,880 4,342,776, 4,513,138, 4,399,276, EP Patent Application Publication Nos . 0 418 099 and 0 088 642, Wani, et al., J. Med. Chem., 1986, 29, 2358, Wani, et al., J. Med. Chem., 1980,23, 554, Wani, et al., J. Med. Chem., 1987, 30, 1774, and Nitta, et al., Proc. 14th International Congr. Chemotherapy., 1985, Anticancer Section I, 28, the entire disclosure of each which is hereby incorporated by reference. The platinum coordination complex, cisplatin, is available under the name Platinol® from Bristol Myers-Squibb Corporation. Useful formulations for cisplatin are described in U.S. Pat. Nos. 5,562,925 and 4,310,515, the entire disclosure of each which is hereby incorporated by reference.

In the method of inhibiting tumor growth which comprises administering stepwise or in physical combination a compound of formula (I) and an antineoplastic agent, the platinum coordination compound, for example cisplatin, can be administered using slow intravenous infusion. The preferred carrier is a dextrose/saline solution containing mannitol. The dose schedule of the platinum coordination compound may be on the basis of from about 1 to about 500 mg per square meter (mg/m²) of body surface area per course of treatment. Infusions of the platinum coordiation compound may be given one to two times weekly, and the weekly treatments may be repeated several times. Using a compound of the camptothecin analog class in a parenteral administration, the course of therapy generally employed is from about 0.1 to about 300.0 mg/m² of body surface area per day for about five consecutive days. Most preferably, the course of therapy employed for topotecan is from about 1.0 to about 2.0 mg/m² of body surface area per day for about five consecutive days. Preferably, the course of therapy is repeated at least once at about a seven day to about a twenty-eight day interval.

The pharmaceutical composition may be formulated with both the compound of formula (I) and the antineoplastic agent in the same container, but formulation in different containers is preferred. When both agents are provided in solution form, they can be contained in an infusion/injection system for simultaneous administration or in a tandem arrangement.

For convenient administration of the compound of formula (I) and the antineoplastic agent at the same or different times, a kit is prepared, comprising, in a single container, such as a box, carton or other container, individual bottles, bags, vials or other containers each having an effective amount of the compound of formula (I) for parenteral administration, as described above, and an effective amount of the antineoplastic agent for parenteral administration, as described above. Such kit can comprise, for example, both pharmaceutical agents in separate containers or the same container, optionally as lyophilized plugs, and containers of solutions for reconstitution. A variation of this is to include the solution for reconstitution and the lyophilized plug in two chambers of a single container, which can be caused to admix prior to use. With such an arrangement, the antineoplastic agent and the compound of this invention may be packaged separately, as in two containers, or lyophilized together as a powder and provided in a single container.

When both agents are provided in solution form, they can be contained in an infusion/injection system for simultaneous administration or in a tandem arrangement. For example, the compound of formula (I) may be in an i.v. injectable form, or infusion bag linked in series, via tubing, to the antineoplastic agent in a second infusion bag. Using such a system, a patient can receive an initial bolus-type injection or infusion of the compound of formula (I) followed by an infusion of the antineoplastic agent.

The compound may be tested in one of several biological assays to determine the concentration of compound which is required to have a given pharmacological effect.

Inhibition of Vitronectin Binding

Solid-Phase [³H]-SK&F-107260 Binding to α_{V}β₃: Human placenta or human platelet α_{V}β₃ (0.1-0.3 mg/mL) in buffer T (containing 2 mM CaCl₂ and 1% octylglucoside) was diluted with buffer T containing 1 mM CaCl₂, 1 mM MnCl₂, 1 mM MgCl₂ (buffer A) and 0.05% NaN₃, and then immediately added to 96-well ELISA plates (Corning, New York, N.Y.) at 0.1 mL per well. 0.1- 0.2μg of α_{V}β₃ was added per well. The plates were incubated overnight at 4° C. At the time of the experiment, the wells were washed once with buffer A and were incubated with 0.1 mL of 3.5% bovine serum albumin in the same buffer for 1 hr at room temperature. Following incubation the wells were aspirated completely and washed twice with 0.2 mL buffer A.

Compounds were dissolved in 100% DMSO to give a 2 mM stock solution, which was diluted with binding buffer (15 mM Tris-HCl (pH 7.4), 100 mM NaCl, 1 mM CaCl₂, 1 mM MnCl₂, 1 mM MgCl₂) to a final compound concentration of 100 μM. This solution is then diluted to the required final compound concentration. Various concentrations of unlabeled antagonists (0.001-100 μM) were added to the wells in triplicates, followed by the addition of 5.0 nM of [³H]-SK&F-107260 (65-86 Ci/mmol).

The plates were incubated for 1 hr at room temperature. Following incubation the wells were aspirated completely and washed once with 0.2 mL of ice cold buffer A in a well-to-well fashion. The receptors were solubilized with 0.1 mL of 1% SDS and the bound [³H]-SK&F-107260 was determined by liquid scintillation counting with the addition of 3 mL Ready Safe in a Beckman LS Liquid Scintillation Counter, with 40% efficiency. Nonspecific binding of [³H]-SK&F- 107260 was determined in the presence of 2 μM SK&F-107260 and was consistently less than 1% of total radio ligand input. The IC₅₀ (concentration of the antagonist to inhibit 50% binding of [³H]-SK&F-107260) was determined by a nonlinear, least squares curve-fitting routine, which was modified from the LUNDON-2 program. The Kᵢ (dissociation constant of the antagonist) was calculated according to the equation: Kᵢ=IC₅₀/(1+L/K_{d}), where L and K_{d} were the concentration and the dissociation constant of [³H]-SK&F-107260, respectively.

The compound of the present invention inhibits vitronectin binding to SK&F 107260 at a concentration of about 0.003 micomolar.

The Compound of this invention is also tested for in vitro and in vivo bone resorption in assays standard in the art for evaluating inhibition of bone formation, such as the pit formation assay disclosed in EP 528 587, which may also be performed using human osteoclasts in place of rat osteoclasts, and the ovarectomized rat model, described by Wronski et al., Cells and Materials 1991, Sup. 1, 69-74.

Vascular Smooth Muscle Cell Migration Assay

Rat or human aortic smooth muscle cells were used. The cell migration was monitored in a Transwell cell culture chamber by using a polycarbonate membrane with pores of 8 um (Costar). The lower surface of the filter was coated with vitronectin. Cells were suspended in DMEM supplemented with 0.2% bovine serum albumin at a concentration of 2.5-5.0×10⁶ cells/mL, and were pretreated with test compound at various concentrations for 20 min at 20° C. The solvent alone was used as control. 0.2 mL of the cell suspension was placed in the upper compartment of the chamber. The lower compartment contained 0.6 mL of DMEM supplemented with 0.2% bovine serum albumin. Incubation was carried out at 37° C. in an atmosphere of 95% air/5% CO₂ for 24 hr. After incubation, the non-migrated cells on the upper surface of the filter were removed by gentle scraping. The filter was then fixed in methanol and stained with 10% Giemsa stain. Migration was measured either by a) counting the number of cells that had migrated to the lower surface of the filter or by b) extracting the stained cells with 10% acetic acid followed by determining the absorbance at 600 nM.

Thyroparathyroidectomized Rat Model

Each experimental group consists of 5-6 adult male Sprague-Dawley rats (250-400 g body weight). The rats are thyroparathyroidectomized (by the vendor, Taconic Farms) 7 days prior to use. All rats receive a replacement dose of thyroxine every 3 days. On receipt of the rats, circulating ionized calcium levels are measured in whole blood immediately after it has been withdrawn by tail venipuncture into heparinized tubes. Rats are included if the ionized Ca level (measured with a Ciba-Corning model 634 calcium pH analyzer) is<1.2 mM/L. Each rat is fitted with an indwelling venous and arterial catheter for the delivery of test material and for blood sampling respectively. The rats are then put on a diet of calcium-free chow and deionized water. Baseline Ca levels are measured and each rat is administered either control vehicle or human parathyroid hormone 1-34 peptide (hPTH 1-34, dose 1.25 ug/kg/h in saline/0.1% bovine serum albumin, Bachem, Ca) or a mixture of hPTH1-34 and test material, by continuous intravenous infusion via the venous catheter using an external syringe pump. The calcemic response of each rat is measured at two-hourly intervals during the infusion period of 6-8 hours.

Human Osteoclast Resorption and Adhesion Assays

Pit resorption and adhesion assays have been developed and standardized using normal human osteoclasts derived from osteoclastoma tissue. Assay 1 was developed for the measurement of osteoclast pit volumes by laser confocal microscopy. Assay 2 was developed as a higher throughput screen in which collagen fragments (released during resorption) are measured by competitive ELISA.

Assay 1 (Using Laser Confocal Microscopy)

Aliquots of human osteoclastoma-derived cell suspensions are removed from liquid nitrogen storage, warmed rapidly at 37° C. and washed x1 in RPMI-1640 medium by centrifugation (1000 rpm, 5 mins at 4° C.).

The medium is aspirated and replaced with murine anti-HLA-DR antibody then diluted 1:3 in RPMI-1640 medium. The suspension is incubated for 30 mins on ice and mixed frequently.

The cells are washed x2 with cold RPMI- 1640 followed by centrifugation (1000 rpm, 5 mins at 4° C.) and the cells are then transferred to a sterile 15 ml centrifuge tube. The number of mononuclear cells are enumerated in an improved Neubauer counting chamber.

Sufficient magnetic beads (5/mononuclear cell), coated with goat anti-mouse IgG (Dynal, Great Neck, N.Y.) are removed from their stock bottle and placed into 5 ml of fresh medium (this washes away the toxic azide preservative). The medium is removed by immobilizing the beads on a magnet and is replaced with fresh medium.

The beads are mixed with the cells and the suspension is incubated for 30 mins on ice. The suspension is mixed frequently.

The bead-coated cells are immobilized on a magnet and the remaining cells (osteoclast-rich fraction) are decanted into a sterile 50 ml centrifuge tube.

Fresh medium is added to the bead-coated cells to dislodge any trapped osteoclasts. This wash process is repeated x10. The bead-coated cells are discarded.

The viable osteoclasts are enumerated in a counting chamber, using fluorescein diacetate to label live cells. A large-bore disposable plastic pasteur pipet is used to add the sample to the chamber.

The osteoclasts are pelleted by centrifugation and the density adjusted to the appropriate number in EMEM medium (the number of osteoclasts is variable from tumor to tumor), supplemented with 10% fetal calf serum and 1.7 g/liter of sodium bicarbonate.

3 ml aliquots of the cell suspension (per compound treatment) are decanted into 15 ml centrifuge tubes. The cells are pelleted by centrifugation.

To each tube, 3 ml of the appropriate compound treatment are added (diluted to 50 uM in the EMEM medium). Also included are appropriate vehicle controls, a positive control (anti-vitronectin receptor murine monoclonal antibody [87MEM1] diluted to 100 ug/ml) and an isotype control (IgG₂ₐ diluted to 100 ug/ml). The samples are incubated at 37° C. for 30 mins.

0.5 ml aliquots of the cells are seeded onto sterile dentine slices in a 48-well plate and incubated at 37° C. for 2 hours. Each treatment is screened in quadruplicate.

The slices are washed in six changes of warm PBS (10 ml/well in a 6-well plate) and then placed into fresh medium containing the compound treatment or control samples. The samples are incubated at 37° C. for 48 hours.

Tartrate Resistant Acid Phosphatase (TRAP) Procedure (Selective Stain for Cells of the Osteoclast Lineage)

The bone slices containing the attached osteoclasts are washed in phosphate buffered saline and fixed in 2% gluteraldehyde (in 0.2M sodium cacodylate) for 5 mins. are then washed in water and are incubated for 4 minutes in TRAP buffer at 37° C. (0.5 mg/ml naphthol AS-BI phosphate dissolved in N,N-dimethylformamide and mixed with 0.25 M citrate buffer (pH 4.5), containing 10 mM sodium tartrate.

Following a wash in cold water the slices are immersed in cold acetate buffer (0.1 M, pH 6.2) containing 1 mg/ml fast red garnet and incubated at 4° C. for 4 minutes.

Excess buffer is aspirated, and the slices are air dried following a wash in water.

The TRAP positive osteoclasts (brick red/ purple precipitate) are enumerated by bright-field microscopy and are then removed from the surface of the dentine by sonication.

Pit volumes are determined using the Nikon/Lasertec ILM21W confocal microscope.

Assay 2 (Using an ELISA Readout)

The human osteoclasts are enriched and prepared for compound screening as described in the initial 9 steps of Assay 1. For clarity, these steps are repeated hereinbelow.

Aliquots of human osteoclastoma-derived cell suspensions are removed from liquid nitrogen strorage, warmed rapidly at 37° C. and washed x1 in RPMI-1640 medium by centrifugation (1000 rpm, 5 mins at 4° C.).

The medium is aspirated and replaced with murine anti-HLA-DR antibody then diluted 1:3 in RPMI-1640 medium. The suspension is incubated for 30 mins on ice and mixed frequently.

The cells are washed x2 with cold RPMI-1640 followed by centrifugation (1000 rpm, 5 mins at 4° C.) and the cells are then transferred to a sterile 15 ml centrifuge tube. The number of mononuclear cells are enumerated in an improved Neubauer counting chamber.

Sufficient magnetic beads (5/mononuclear cell), coated with goat anti-mouse IgG (Dynal, Great Neck, N.Y.) are removed from their stock bottle and placed into 5 ml of fresh medium (this washes away the toxic azide preservative). The medium is removed by immobilizing the beads on a magnet and is replaced with fresh medium.

The beads are mixed with the cells and the suspension is incubated for 30 mins on ice. The suspension is mixed frequently.

The bead-coated cells are immobilized on a magnet and the remaining cells (osteoclast-rich fraction) are decanted into a sterile 50 ml centrifuge tube.

Fresh medium is added to the bead-coated cells to dislodge any trapped osteoclasts. This wash process is repeated x10. The bead-coated cells are discarded.

The viable osteoclasts are enumerated in a counting chamber, using fluorescein diacetate to label live cells. A large-bore disposable plastic pasteur pipet is used to add the sample to the chamber.

The osteoclasts are pelleted by centrifugation and the density adjusted to the appropriate number in EMEM medium (the number of osteoclasts is variable from tumor to tumor), supplemented with 10% fetal calf serum and 1.7 g/liter of sodium bicarbonate.

In contrast to the method described above in Assay 1, the compounds are screened at 4 doses to obtain an IC₅₀, as outlined below:

The osteoclast preparations are preincubated for 30 minutes at 37° C. with test compound (4 doses) or controls.

They are then seeded onto bovine cortical bone slices in wells of a 48-well tissue culture plate and are incubated for a further 2 hours at 37° C.

The bone slices are washed in six changes of warm phosphate buffered saline (PBS), to remove non-adherent cells, and are then returned to wells of a 48 well plate containing fresh compound or controls.

The tissue culture plate is then incubated for 48 hours at 37° C.

The supernatants from each well are aspirated into individual tubes and are screened in a competitive ELISA that detects the c-telopeptide of type I collagen which is released during the resorption process. This is a commercially available ELISA (Osteometer, Denmark) that contains a rabbit antibody that specifically reacts with an 8-amino acid sequence (Glu-Lys-Ala-His- Asp-Gly-Gly-Arg) that is present in the carboxy-terminal telopeptide of the a1-chain of type I collagen. The results are expressed as % inhibition of resorption compared to a vehicle control.

Human Osteoclast Adhesion Assay

The human osteoclasts are enriched and prepared for compound screening as described above in the initial 9 steps of Assay 1. For clarity, these steps are repeated hereinbelow.

Aliquots of human osteoclastoma-derived cell suspensions are removed from liquid nitrogen strorage, warmed rapidly at 37° C. and washed x1 in RPMI-1640 medium by centrifugation (1000 rpm, 5 mins at 4° C.).

The medium is aspirated and replaced with murine anti-HLA-DR antibody then diluted 1:3 in RPMI-1640 medium. The suspension is incubated for 30 mins on ice and mixed frequently.

The cells are washed x2 with cold RPMI-1640 followed by centrifugation (1000 rpm, 5 mins at 4° C.) and the cells are then transferred to a sterile 15 ml centrifuge tube. The number of mononuclear cells are enumerated in an improved Neubauer counting chamber.

Sufficient magnetic beads (5/mononuclear cell), coated with goat anti-mouse IgG (Dynal, Great Neck, N.Y.) are removed from their stock bottle and placed into 5 ml of fresh medium (this washes away the toxic azide preservative). The medium is removed by immobilizing the beads on a magnet and is replaced with fresh medium.

The beads are mixed with the cells and the suspension is incubated for 30 mins on ice. The suspension is mixed frequently.

The bead-coated cells are immobilized on a magnet and the remaining cells (osteoclast-rich fraction) are decanted into a sterile 50 ml centrifuge tube.

Fresh medium is added to the bead-coated cells to dislodge any trapped osteoclasts. This wash process is repeated x10. The bead-coated cells are discarded.

The viable osteoclasts are enumerated in a counting chamber, using fluorescein diacetate to label live cells. A large-bore disposable plastic pasteur pipet is used to add the sample to the chamber.

The osteoclasts are pelleted by centrifugation and the density adjusted to the appropriate number in EMEM medium (the number of osteoclasts is variable from tumor to tumor), supplemented with 10% fetal calf serum and 1.7g/liter of sodium bicarbonate.

Osteoclastoma-derived osteoclasts are preincubated with compound (4 doses) or controls at 37° C. for 30 minutes.

The cells are then seeded onto osteopontin-coated slides (human or rat osteopontin, 2.5 ug/ml) and incubated for 2 hours at 37° C.

Non adherent cells are removed by washing the slides vigorously in phosphate buffered saline and the cells remaining on the slides are fixed in acetone.

The osteoclasts are stained for tartrate-resistant acid phosphatase (TRAP), a selective marker for cells of this phenotype (see steps 15 -17), and are enumerated by light microscopy. The results are expressed as % inhibition of adhesion compared to a vehicle control.

Cell Adhesion Assay

Cells and Cell Culture

Human embryonic kidney cells (HEK293 cells) were obtained from ATCC (Catalog No. CRL 1573). Cells were grown in Earl's minimal essential medium (EMEM) medium containing Earl's salts, 10% fetal bovine serum, 1% glutamine and 1% Penicillin-Steptomycin.

Constructs and Transfections

A 3.2 kb EcoRI-KpnI fragment of the av subunit and a 2.4 kb XbaI- XboI fragment of the β₃ subunit were inserted into the EcoRI - EcoRV cloning sites of the pCDN vector (Aiyar et al., 1994 ) which contains a CMV promoter and a G418 selectable marker by blunt end ligation. For stable expression, 80×10⁶ HEK 293 cells were electrotransformed with α_{V}+β₃ constructs (20 μg DNA of each subunit) using a Gene Pulser (Hensley et al., 1994) and plated in 100 mm plates (5×10⁵ cells/plate). After 48 hr, the growth medium was supplemented with 450 μg/mL Geneticin (G418 Sulfate, GIBCO-BRL, Bethesda, Md.). The cells were maintained in selection medium until the colonies were large enough to be assayed.

Immunocytochemical Analysis of Transfected Cells

To determine whether the HEK 293 transfectants expressed the vitronectin receptor, the cells were immobilized on glass microscope slides by centrifugation, fixed in acetone for 2 min at room temperature and air dried. Specific reactivity with 23C6, a monoclonal antibody specific for the α_{V}β₃ complex was demonstrated using a standard indirect immunofluorescence method.

Cell Adhesion Studies

Corning 96-well ELISA plates were precoated overnight at 4° C. with 0.1 mL of human vitronectin (0.2 μg/mL in RPMI medium). At the time of the experiment, the plates were washed once with RPMI medium and blocked with 3.5% BSA in RPMI medium for 1 hr at room temperature. Transfected 293 cells were resuspended in RPMI medium, supplemented with 20 mM Hepes, pH 7.4 and 0.1% BSA at a density of 0.5×10⁶ cells/mL. 0.1 mL of cell suspension was added to each well and incubated for 1 hr at 37° C., in the presence or absence of various α_{V}β₃ antagonists. Following incubation, 0.025 mL of a 10% formaldehyde solution, pH 7.4, was added and the cells were fixed at room temperature for 10 min. The plates were washed 3 times with 0.2 mL of RPMI medium and the adherent cells were stained with 0.1 mL of 0.5% toluidine blue for 20 min at room temperature. Excess stain was removed by extensive washing with deionized water. The toluidine blue incorporated into cells was eluted by the addition of 0.1 mL of 50% ethanol containing 50 mM HCl. Cell adhesion was quantitated at an optical density of 600 nm on a microtiter plate reader (Titertek Multiskan MC, Sterling, Va.).

Solid-Phase α_{V}β₅ Binding Assay:

The vitronectin receptor α_{V}β₅ was purified from human placenta. Receptor preparation was diluted with 50 mM Tris-HCl, pH 7.5, 100 mM NaCl, 1 mM CaCl₂, 1 mM MnCl₂, 1 mM MgCl₂ (buffer A) and was immediately added to 96-well ELISA plates at 0.1 ml per well. 0.1-0.2 μg of α_{V}β₃ was added per well. The plates were incubated overnight at 4° C. At the time of the experiment, the wells were washed once with buffer A and were incubated with 0.1 ml of 3.5% bovine serum albumin in the same buffer for 1 hr at room temperature. Following incubation the wells were aspirated completely and washed twice with 0.2 ml buffer A.

In a [³H]-SK&F- 107260 competition assay, various concentrations of unlabeled antagonists (0.001-100 μM) were added to the wells, followed by the addition of 5.0 nM of [³H]-SK&F-107260. The plates were incubated for 1 hr at room temperature. Following incubation the wells were aspirated completely and washed once with 0.2 ml of ice cold buffer A in a well-to-well fashion. The receptors were solubilized with 0.1 ml of 1% SDS and the bound [³H]-SK&F-107260 was determined by liquid scintillation counting with the addition of 3 ml Ready Safe in a Beckman LS 6800 Liquid Scintillation Counter, with 40% efficiency. Nonspecific binding of [³H]-SK&F-107260 was determined in the presence of 2 μM SK&F-107260 and was consistently less than 1% of total radio ligand input. The IC₅₀ (concentration of the antagonist to inhibit 50% binding of [³H]-SK&F-107260) was determined by a nonlinear, least squares curve-fitting routine, which was modified from the LUNDON-2 program. The Kᵢ (dissociation constant of the antagonist) was calculated according to Cheng and Prusoff equation: Kᵢ=IC₅₀/(1+L/K_{d}), where L and K_{d} were the concentration and the dissociation constant of [³H]-SK&F-107260, respectively.

### Inhibition of RGD-Mediated GPIIb-IIIa Binding

Purification of GPIIb-IIIa

Ten units of outdated, washed human platelets (obtained from Red Cross) were lyzed by gentle stirring in 3% octylglucoside, 20 mM Tris-HCl, pH 7.4, 140 MM NaCl, 2 mM CaCl₂ at 4° C. for 2 h. The lysate was centrifuged at 100,000 g for 1 h. The supernatant obtained was applied to a 5 mL lentil lectin sepharose 4B column (E. Y. Labs) preequilibrated with 20 mM Tris-HCl, pH 7.4, 100 mM NaCl, 2 mM CaCl₂, 1% octylglucoside (buffer A). After 2 h incubation, the column was washed with 50 mL cold buffer A. The lectin-retained GPIIb-IIIa was eluted with buffer A containing 10% dextrose. All procedures were performed at 4° C. The GPIIb-IIIa obtained was >95% pure as shown by SDS polyacrylamide gel electrophoresis.

Incorporation of GPIIb-IIIa in Liposomes.

A mixture of phosphatidylserine (70%) and phosphatidylcholine (30%) (Avanti Polar Lipids) were dried to the walls of a glass tube under a stream of nitrogen. Purified GPIIb-IIIa was diluted to a final concentration of 0.5 mg/mL and mixed with the phospholipids in a protein:phospholipid ratio of 1:3 (w:w). The mixture was resuspended and sonicated in a bath sonicator for 5 min. The mixture was then dialyzed overnight using 12,000-14,000 molecular weight cutoff dialysis tubing against a 1000-fold excess of 50 mM Tris-HCl, pH 7.4, 100 mM NaCl, 2 mM CaCl2 (with 2 changes). The GPIIb-IIIa-containing liposomes wee centrifuged at 12,000 g for 15 min and resuspended in the dialysis buffer at a final protein concentration of approximately 1 mg/mL. The liposomes were stored at −70C. until needed.

Competitive Binding to GPIIb-IIIa

The binding to the fibrinogen receptor (GPIIb-IIIa) was assayed by an indirect competitive binding method using [³H]-SK&F-107260 as an RGD-type ligand. The binding assay was performed in a 96-well filtration plate assembly (Millipore Corporation, Bedford, Mass.) using 0.22 um hydrophilic durapore membranes. The wells were precoated with 0.2 mL of 10 μg/mL polylysine (Sigma Chemical Co., St. Louis, Mo.) at room temperature for 1 h to block nonspecific binding. Various concentrations of unlabeled benzazepines were added to the wells in quadruplicate. [³H]-SK&F-107260 was applied to each well at a final concentration of 4.5 nM, followed by the addition of 1 μg of the purified platelet GPIIb-IIIa-containing liposomes. The mixtures were incubated for 1 h at room temperature. The GPIIb-IIIa-bound [3H]-SK&F- 107260 was separated from the unbound by filtration using a Millipore filtration manifold, followed by washing with ice-cold buffer (2 times, each 0.2 mL). Bound radioactivity remaining on the filters was counted in 1.5 mL Ready Solve (Beckman Instruments, Fullerton, Calif.) in a Beckman Liquid Scintillation Counter (Model LS6800), with 40% efficiency. Nonspecific binding was determined in the presence of 2 pM unlabeled SK&F-107260 and was consistently less than 0.14% of the total radioactivity added to the samples. All data points are the mean of quadruplicate determinations.

Competition binding data were analyzed by a nonlinear least-squares curve fitting procedure. This method provides the IC50 of the antagonists (concentration of the antagonist which inhibits specific binding of [³H]-SK&F-107260 by 50% at equilibrium). The IC50 is related to the equilibrium dissociation constant (Ki) of the antagonist based on the Cheng and Prusoff equation: Ki=IC_{50/}(1+L/Kd), where L is the concentration of [3H]-SK&F-107260 used in the competitive binding assay (4.5 nM), and Kd is the dissociation constant of [3H]-SK&F-107260 which is 4.5 nM as determined by Scatchard analysis.

The compound of this invention has an affinity for the vitronectin receptor relative to the fibrinogen receptor of greater than 10:1. This compound has a ratio of activity of greater than 100:1.

The efficacy of the compound of formula (I) alone or in combination with an antineoplastic agent may be determined using several transplantable mouse tumor models. See U.S. Pat. Nos. 5,004,758 and 5,633,016 for details of these models

The examples which follow are intended in no way to limit the scope of this invention, but are provided to illustrate how to make and use the compound of this invention. Many other embodiments will be readily apparent to those skilled in the art.

### EXAMPLES

### General

¹H nuclear magnetic resonance (NMR) spectra were recorded at either 250 or 400 MHz. Chemical shifts are reported in parts per million (δ) downfield from the internal standard tetramethylsilane (TMS). Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. CDCl₃ is deuteriochloroform, DMSO-d₆ is hexadeuteriodimethylsulfoxide, and CD₃OD is tetradeuteriomethanol. Infrared (IR) spectra were recorded in transmission mode, and band positions are reported in inverse wavenumbers (cm⁻¹). Mass spectra were obtained using electrospray (ES) ionization techniques. Elemental analyses were performed by Quantitative Technologies Inc., Whitehouse, N.J. Melting points were taken on a Thomas-Hoover melting point apparatus and are uncorrected. All temperatures are reported in degrees Celsius. Analtech Silica Gel G F and E. Merck Silica Gel 60 F-254 thin layer plates were used for thin layer chromatography. Both flash and gravity chromatography were carried out on E. Merck Kieselgel 60 (230-400 mesh) silica gel. Analytical and preparative HPLC were carried out on Rainin or Beckman chromatographs. ODS refers to an octadecylsilyl derivatized silica gel chromatographic support. 5μ Apex-ODS indicates an octadecylsilyl derivatized silica gel chromatographic support having a nominal particle size of 5μ, made by Jones Chromatography, Littleton, Colo. YMC ODS-AQ® is an ODS chromatographic support and is a registered trademark of YMC Co. Ltd., Kyoto, Japan. PRP-1® is a polymeric (styrene-divinylbenzene) chromatographic support, and is a registered trademark of Hamilton Co., Reno, Nev. Celite® is a filter aid composed of acid-washed diatomaceous silica, and is a registered trademark of Manville Corp., Denver, Colo.

### Example 1

### Preparation of (S)-8-[3-(4-methylpyridin-2-ylamino)-1-propyloxy]-3-oxo-2-[4-(trifluoromethyl)benzyl]-2, 3, 4, 5-tetrahydro-1H-2-benzazepine-4-acetic acid

### Preparation 1

### Preparation of methyl (±)-8-hydroxy-3-oxo-2, 3, 4, 5-tetrahydro-1H-2-benzazepine-4-acetate

a) 4-Bromo-3-bromomethylanisole

A mixture of 2-bromo-5-methoxytoluene (20 g, 0.10 mol), N-bromosuccinimide (19.6 g, 0.11 mol), benzoyl peroxide (1 g, 4 mmol), and methylene chloride (200 mL) was irradiated for 18 hr with a flood lamp to effect gentle reflux. The mixture was then cooled to −10° C. for several hours and the solution was decanted away from the precipitated succinimide. The solution was concentrated and the residue was crystallized from chloroform/hexane to give the title compound (19.7 g, 70%) as pale yellow prisms: ¹H NMR (CDCl₃) δ 7.45 (d, J=8.9 Hz, 1H), δ .99 (d, J=3 Hz, 1H), 6.74 (dd, J=8.9, 3 Hz, 1 H), 4.55 (s, 2 H) 3.80 (s, 3 H).

b) 3-Bis(tert-butoxycarbonyl)aminomethyl-4-bromoanisole

A mixture of 4-bromo-3-bromomethylanisole (24 g, 86 mmol) and potassium di-tert-butyl iminodicarboxylate (24 g, 94 mmol) in dimethylformamide (200 mL) was stirred under argon at room temperature for 18 hr. The reaction was then concentrated under vacuum and the residue was partitioned between ethyl acetate and water. The organic phase was washed with water and brine, dried(MgSO₄), and concentrated. The residue was recrystallized from hexane to give the title compound (15 g, 42%) as a white solid: ¹H NMR (CDCl₃) δ 7.40 (d, J=8.6 Hz, 1 H)), 6.68 (m, 2 H), 4.81(s, 2 H), 3.74 (s, 3 H), 1.44 (s, 18 H).

c) Methyl (±)-3-carbomethoxy-4-[2-bis(tert-butoxycarbonyl)aminomethyl-4-methoxyphenyl]-3-butenoate

A 500 mL flask was charged with 3-bis(tert-butoxycarbonyl)aminomethyl-4-bromoanisole(15 g, 36 mmol), dimethyl itaconate (7.5 g, 47 mmol), tri-o-tolylphosphine (1 g, 3 mol), palladium acetate (0.4 g, 2 mmol), diisopropylethylamine (12.8 mL, 72 mmol), and propionitrile (150 mL). The mixture was purged with argon (several evacuation/argon flush cycles), then was heated to reflux under argon for 1 hr. The reaction was allowed to cool to RT, then was poured into ice-cold ethyl ether (500 mL). The resulting precipitate was removed by filtration and the filtrate was concentrated. The residue was purified by chromatography on silica gel (10% -20% ethyl acetate in hexane) to give the title compound (11.8 g, 66%) as a pale yellow oil: ¹H NMR (CDCl₃) δ 7.94 (s, 1 H), 7.15 (d, J=8.1 Hz, 1H)), 6.77 (d, J=8.1 Hz, 1H), 6.76 (s, 1H), 4.73 (s, 2 H), 3.81 (s, 3 H), 3.79 (s, 3 H), 3.71 (s, 3 H), 3.38 (s, 2 H), 1.45 (s, 18 H).

d) Methyl (±)-3-carbomethoxy-4-[2-bis(tert-butoxycarbonyl)aminomethyl-4-methoxyphenyl]butanoate

A pressure vessel charged with methyl (±)-3-carbomethoxy4-[2-bis(tert-butoxycarbonyl)aminomethyl-4-methoxyphenyl]-3-butenoate (11.8 g), ethyl acetate (120 mL), and 10% palladium on charcoal (1 g) was shaken under 45 psi of hydrogen for 18 hr. The mixture was then filtered and the filtrate was concentrated to give the title compound (12 g, 100%) as a colorless oil: ¹H NMR (CDCl₃) δ 7.00 (d, J=8.2 Hz, 1 H), 6.71 (m, 2 H), 4.81 (s, 2 H), 3.75 (s, 3 H), 3.66 (s,3 H), 3.63 (s, 3 H), 3.05 (m, 2 H), 2.73 (m, 2 H), 2.42 (dd, J=16.0, 4.8 Hz, 1H), 1.44 (s, 18 H).

e) Methyl (±)-3-carbomethoxy-4-[2-(aminomethyl)4methoxyphenyl]butanoate

A solution of methyl (±)-3-carbomethoxy-4-[2-bis(tert-butoxycarbonyl)aminomethyl-4-methoxyphenyl]butanoate (12 g) in chloroform (100 mL) and trifluoroacetic acid (50 mL) was stirred under argon at room temperature for 4 hr. The solution was then concentrated under vacuum to give the title compound (10 g, 100%) as a viscous oil: MS (ES) m/e 296.2 (M+H)⁺.

f) Methyl (±)-8-methoxy-3-oxo-2, 3, 4, 5-tetrahydro- 1H-2-benzazepine-4-acetate

A solution of methyl (±)-3-carbomethoxy4-[2-(aminomethyl)-4-methoxyphenyl]butanoate (10 g, 24 mmol) and triethylamine (17 mL, 120 mmol) in toluene (100 mL) was heated at reflux for 18 hr. The reaction was then concentrated and the residue was partitioned between ethyl acetate and water. The aqueous layer was extracted twice with ethyl acetate and the combined organic extracts were washed with brine, dried (MgSO₄), and concentrated to afford the title compound (4.8 g, 76%) as tan solid: MS (ES) m/e 264.2 (M+H)⁺.

g) Methyl (±)-8-hydroxy-3-oxo-2, 3, 4, 5-tetrahydro-1H-2-benzazepine-4-acetate

Anhydrous aluminum chloride (7.6 g, 57 mmol) was added portionwise to a stirred solution of methyl (±)-8-methoxy-3-oxo-2, 3, 4, 5-tetrahydro-1H-2-benzazepine-4-acetate (3.0 g, 11 mmol) and ethanethiol (4.2 mL, 57 mmol) in methylene chloride (100 mL) at 0° C. under argon. The resulting mixture was allowed to warm to room temperature and stir overnight, then was concentrated. The residue was triturated with ice-water, and the resulting solid was collected by filtration and dried to give the title compound (2.64 g, 91%) as an off-white solid: MS (ES) m/e 250.2 (M+H)⁺.

### Preparation 2

### HPLC separation of the enantiomers of methyl (±)-8-hydroxy-3-oxo-2, 3, 4, 5-tetrahydro-1H-2-benzazepine-4-acetate

a) Methyl (R)-(+)-8-hydroxy-3-oxo-2, 3, 4, 5-tetrahydro-1H-2-benzazepine-4-acetate and methyl (S)-(−)-8-hydroxy-3-oxo-2, 3, 4, 5-tetrahydro-1H-2-benzazepine-4-acetate

Methyl (±)-8-hydroxy-3-oxo-2, 3, 4, 5-tetrahydro-1H-2-benzazepine-4-acetate was resolved into its enantiomers by chiral HPLC using the following conditions: Diacel Chiralpak AS® column (21.2×250 mm), EtOH mobile phase, 7 mL/min flowrate, uv detection at 254 nm, 70 mg injection; t_{R} for methyl (R)-(+)-8-hydroxy-3-oxo-2, 3, 4, 5-tetrahydro-1H-2-benzazepine-4-acetate=21.5 min; t_{R} for methyl (S)-(−)-8-hydroxy-3-oxo-2, 3, 4, 5-tetrahydro-1H-2-benzazepine-4-acetate=39.1 min.

### Preparation 3

### HPLC separation of the enantiomers of methyl (±)-8-methoxy-3-oxo-2, 3, 4, 5-tetrahydro-1H-2-benzazepine-4-acetate

a) Methyl (R)-(+)-8-methoxy-3-oxo-2, 3, 4, 5-tetrahydro-1H-2-benzazepine4-acetate and methyl (S)-(−)-8-methoxy-3-oxo-2, 3, 4, 5-tetrahydro-1H-2-benzazepine-4-acetate

Methyl (±)-8-methoxy-3-oxo-2, 3, 4, 5-tetrahydro-1H-2-benzazepineacetate was resolved into its enantiomers by chiral HPLC using the following conditions: Diacel Chiralpak AS® column (21.2×250 mm), CH₃CN mobile phase, 15 mL/min flowrate, uv detection at 254 nm, 500 mg injection; t_{R} for methyl (R)-(+)-8-methoxy-3-oxo-2, 3, 4, 5-tetrahydro-1H-2-benzazepine-4-acetate=10.2 min; t_{R} for methyl (S)-(−)-8-methoxy-3-oxo-2, 3, 4, 5-tetrahydro-1H-2-benzazepine-4-acetate =19.0 min.

### Preparation 4

### Demethylation of methyl (S)-8-methoxy-3-oxo-2, 3, 4, 5-tetrahydro-1H-2-benzazepine-4-acetate

a) Methyl (S)-8-hydroxy-3-oxo-2, 3, 4, 5-tetrahydro-1H-2-benzazepine-4-acetate

A solution of methyl (S)-8-methoxy-3-oxo-2, 3, 4, 5-tetrahydro-1H-2-benzazepine-4-acetate (15.0 g, 0.057 mole) in CHCl₃ (160 mL) was added dropwise over 30 min to a solution of boron tribromide (20.53 mL, 0.217 mole) in CHCl₃ (160 mL) at −8° C. under argon, maintaining the temperature between −5° C. and 0° C. The reaction mixture was stirred at ca. −8° C. for 30 min and then MeOH (200 mL) was added, dropwise initially, maintaining the temperature at ca. 0° C. The reaction mixture was concentrated to give a viscous oil which was reconcentrated from MeOH (100 mL). The oil was dissolved in H₂O/MeOH and a small amount of dark solid was removed by filtration. The filtrate was neutralized (to pH 7) with 50% sodium hydroxide, depositing a white solid. The suspension pH was adjusted to 4.5 by the addition of a small amount of acetic acid and the solid was collected and dried in vacuum to give afford the title compound (9.7 g, 68%). The product was assayed for chiral purity by HPLC: Chiralpak AS® column (4.6×50 mm), 100% EtOH mobile phase, 0.5 mL/min flow rate, uv detection at 215 nm; t_{R}=7.5 min (S-enantiomer, 99%); tR=4.4 min (R-enantiomer, 1%).

### Preparation 5

### Preparation of methyl (S)-8-hydroxy-3-oxo-2-[4-(trifluoromethyl)benzyl]-2, 3, 4, 5-tetrahydro-1H -2-benzazepine-4-acetate via alkylation of methyl (S)8-hydroxy-3-oxo-2, 3, 4, 5-tetrahydro-1H-2-benzazepine-4-acetate

a) Methyl (S)-3-oxo-S-[4-(trifluoromethyl)benzyloxyl-2-[4-(trifluoromethyl)benzyl]-2, 3, 4, 5-tetrahydro- H-2-benzazepine-4-acetate

To a solution of methyl (S)-8-hydroxy-3-oxo-2, 3, 4, 5-tetrahydro-2-1H-benzazepine-4-acetate (0.31 g, 1.24 mmol) and 4-(trifluoromethyl)benzyl bromide (0.89 g, 3.72 mmol) in DMF (10 mL) was added NaH (60% suspension in oil, 0.11 g, 2.75 mmol). After stirring at RT for 4 h, the bulk of the DMF was removed under vacuum. The residue was partitioned between sat. NaHCO₃ and EtOAc. The aqueous phase was extracted with EtOAc and the combined organic extracts were washed with sat. NaCl, dried over Na₂SO₄ and concentrated to give a clear oil (0.90 g). Radial chromatography (5% acetone/CH₂Cl₂, silica gel, 6 m plate) gave the title compound (0.53 g) as a white foam. MS (ES) m/e 566.1 (M+H)⁺.

b) Methyl (S)-8-hydroxy-3-oxo-2-[4-(trifluoromethyl)benzyl]-2, 3, 4, 5-tetrahydro-1H-2-benzazepine4-acetate

A Parr hydrogenation flask was charged with methyl (S)-3-oxo-8-[4-(trifluoromethyl)benzyloxy]-2-H4-(trifluoromethyl)benzyl]-2, 3, 4, 5-tetrahydro-1H-2-benzazepine-4-acetate (0.78 g, 1.38 mmol) and Pearlman's catalyst (20 mg) in MeOH (20 mL). After hydrogenating at 50 psi for 24 h, the reaction vessel was vented and the catalyst was removed by filtration. Removal of solvent gave a white foam (0.60 g). Radial chromatography (5% acetone/CH₂Cl₂, silica gel, 6 m plate) gave the title compound (0.42 g) as a white foam. ¹H NMR (250 MHz, CDCl₃) d 7.50 (d, J=8.5 Hz, 2H), 7.23 (d, J=8.5 Hz, 2H), 6.90 (d, J=7.5 Hz, 1H), 6.67 (dd, J=7.5, 3.4 Hz, 1H), 6.39 (d, J=3.4 Hz, 1H), 5.05 (m, 2 H), 4.35 (d, J=15.4 Hz, 1H), 3.85 (m, 1H), 3.70 (s, 3H), 3.60 (m, 1H), 2.95 (m, 4H), 2.45 (dd, J=17. 1, 5.1 Hz, 1H).

### Preparation 6

### Preparation of methyl (S)-8-hydroxy-3-oxo-2-[4-(trifluoromethyl)benzyl]-2, 3, 4, 5-tetrahydro-1H-2-benzazepine-4-acetate via enantioselective synthesis

a) 4-Bromo-3-bromomethylanisole

To a stiffed solution of 4-bromo-3-methylanisole (100 g, 497 mmol) in dry dichloromethane (500 mL) was added N-bromosuccinimide (97 g, 545 mmol) followed by benzoyl peroxide (6 g, 25 mmol). The reaction was gently refluxed with a 150 watt flood lamp with reflector placed approximately 12 inches from the reaction flask. After 24 h the reaction was concentrated by rotary evaporation to half its volume and allowed to sit for 4 h. The white precipitate which formed was filtered off and rinsed with a small volume of dichloromethane. The filtrate was concentrated to dryness and the remaining solid was triturated with hexanes and filtered. Drying under vacuum gave the title compound (100.25 g, 72%) as white needles: GC t_{R}=6.56 min (HP 530 μm×20 m methylsilicone column, He carrier flow 20 mL/min, 100° C. initial temp., 1 min initial time, 10° C./min rate, 200° C. final temp., 1 min final time); ¹H NMR (400 MHz, CDCl₃) δ 7.44 (d, J=10 Hz, 1 H), 6.99 (d, J=3 Hz, 1H), 6.73 (dd, 1H), 4.55 (s, 2H), 3.80 (s, 3H).

b) 3-[N-(4-Trifluoromethylbenzyl)aminomethyl]-4bromoanisole

To a stirred solution of 4-bromo-3-bromomethylanisole (35 g, 125 mmol) in anhydrous DMSO (50 mL) and dry THF (50 mL) was added 4-trifluoromethylbenzylamine (30 g, 171 mmol) followed by triethylamine (18 mL, 129 mmol). After stirring for 18 h at RT the reaction was concentrated, diluted with aqueous 1 N NaOH (250 mL) and extracted with Et₂O (2×250 mL). The combined organic layers were washed with brine, dried (Na₂SO₄), and concentrated to dryness. The residue which remained was purified by flash chromatography on silica gel (10 to 20% EtOAc/CHCl₃) to give the title compound (34.17 g, 73%): TLC (20% EtOAc/CHCl₃) R_{f}0.63; ¹H NMR (400 MHz, CDCl₃) δ 7.59 (d, J=8.2 Hz, 2H), 7.49 (d, J=8.2 Hz, 2H), 7.43 (d, J=8.6 Hz, 1H), 6.96 (d, J=3.1 Hz, 1H), 6.70 (dd, 1H), 3.86 (s, 2H), 3.84 (s, 2H), 3.79 (s, 3H), 1.75 (br s, 1H).

c) 3-[N-(tert-Butoxycarbonyl)-N-(4-trifluoromethylbenzyl)aminomethyl]-4bromoanisole

To a stirred solution of 3-[N-(4-trifluoromethylbenzyl)aminomethyl]-4-bromoanisole (34.17 g, 91 mmol) in dry THF (100 mL) was added di-tert-butyl dicarbonate (22 g, 101 mmol). The reaction was stirred under argon for 18 h (vigorous gas evolution was observed). Concentration and silica gel chromatography (5 to 10% EtOAc/hexane) gave the title compound (41.09 g, 95%) as a clear oil: TLC (silica, 20% EtOAc/hexane) R_{f} 0.44; ¹H NMR (400 MHz, CDCl₃) δ 7.57 (d, J=8.3 Hz, 2H), 7.40 (d, J=8.3 Hz, 2H), 7.39-7.33 (m, 2H), 6.83 and 6.72 (2 s, 1H), 6.71 (dd, 1H), 4.54 and 4.50 (2 s, 2H), 4.43 (s, 2H), 3.75 (s, 3H), 1.47 (s, 9H).

d) Methyl 2-[N-(tert-butoxycarbonyl)-N-(4-trifluoromethylbenzyl)amninomethyl]-4-methoxycinnamate

A solution of 3-[N-(tert-butoxycarbonyl)-N-(4-trifluoromethylbenzyl)aminomethyl]-4-bromoanisole (37.08 g, 78 mmol), methyl acrylate (35 mL, 390 mmol), palladium acetate (0.88 g, 3.9 mmol), tri-o-tolylphosphine (2.38 g, 7.8 mol), and diisopropylethylamine (31 mL, 178 mmol) in acetonitrile (200 mL) was deoxygenated (3 evacuation/argon purge cycles), then was heated to reflux under argon (oil bath set at 80° C.). After 6 hr additional palladium acetate (0.88 g, 3.9 mmol) and tri-o-tolylphosphine ((2.38 g, 7.8 mmol) were added and the reaction was stirred under reflux for an additional 18 h. The reaction was concentrated to dryness, and the residue was taken up in 1:1 Et₂O/petroleum ether (300 mL) and allowed to stand for 4 h. A gray-colored precipitate was filtered off and washed with a small volume of 1: 1 Et₂O/petroleum ether (100 mL). The orangish-red filtrate was concentrated and purified by flash chromatography on silica gel (15% ethyl acetate/hexanes). The resulting residue was taken up in hexane, and the mixture was allowed to stand for several hr, then was filtered to remove a yellow precipitate. Concentration of the filtrate left the title compound (34.52 g, 92%) as a thick yellow oil: TLC (silica, 20% EtOAc/hexanes) R_{f}0.45; ¹H NMR (400 MHz, CDCl₃) δ 7.80 (br s, 1H); 7.57 (d, J=8.1 Hz, 2H), 7.53 (d, J=8.6 Hz, 1H), 7.29 (br s, 2H), 6.83 (dd, 1H), 6.72 (br s, 1H), 6.23 (d, J=15.7 Hz, 1H), 4.58 and 4.53 (2 br s, 2H), 4.46 and 4.37 (2 br s, 2H), 3.80 (s, 3H), 3.77 (s, 3H), 1.49 (s, 9H).

e) Methyl 2-[N-(tert-butoxycarbonyl)-N-(4-trifluoromethylbenzyl)aminomethyl]-4-methoxydihydrocinnamate

To 10% Pd/C (5 g, 4.7 mmol, prewetted with DMF) was added a solution of methyl 2-[N-(tert-butoxycarbonyl)-N-(4-trifluoromethylbenzyl)aminomethyl]-4-methoxycinnamate (34.52 g, 72 mmol) in methanol (100 mL). The mixture was shaken under hydrogen (50 psi) in a Parr apparatus for 7 hr, then was filtered through a pad of celite® to remove the catalyst. The filtrate was concentrated to afford the title compound (34.15 g, 98%) as a colorless oil: ¹H NMR (400 MHz, CDCl₃) δ 7.58 (d, J=8.1 Hz, 2H), 7.31 (br s, 2H), 7.09 (d, J=8.4 Hz, 1H), 6.76 (dd, 1H), 6.66 (s, 1H), 4.47 (br s, 2H), 4.40 (br s, 21H), 3.76 (s, 3H), 3.63 (s, 3H), 2.79 (br s, 2H), 2.47 (t, 2H), 1.48 (s, 9H).

f) 2-[N-(tert-Butoxycarbonyl)-N-(4-trifluoromethylbenzyl)aminomethyl]-4-methoxydihydrocinnamic acid

To a stirred solution of 2-[N-(tert-butoxycarbonyl)-N-(4-trifluorobenzyl)aminomethyl]-4-methoxydihydrocinnamic acid (34.15 g, 71 mmol) in dioxane (150 mL) was added aqueous 1 N NaOH (85 mL, 85 mmol). The cloudy reaction was stirred at RT for 4 h. The resulting homogeneous solution was neutralized with aqueous 1 N HCl (85 mL, 85 mmol) and extracted with ethyl acetate (2×250 mL). The combined organic layers were washed with brine (250 mL), dried (MgSO₄) and concentrated to give the title compound (34.60 g, 100%) as a thick clear oil: TLC (95:4:1 CHCl₃/MeOH/HOAc) R_{f}0.49; ¹H NMR (400 MHz, CDCl₃) δ 7.58 (d, J=8.1 Hz, 2H), 7.30 (br s, 2H), 7.09 (d, J=8.4 Hz, 1H), 6.78 (dd, 1H), 6.65 (d, J=2.6 Hz, 1H), 4.47 (br s, 2H), 4.42 (br s, 2H), 3.76 (s, 3H), 2.81 (br s, 2H), 2.53 (t, 2H), 1.47 (s, 9H).

g) (R)-4-Benzyl-2-oxazol idinonyl 2-[N-(tert-butoxycarbonyl)-N-(4-trifluoromethylbenzyl)aminomethyl]-4-methoxydihydrocinnamide

To a stirred solution of 2-[N-(tert-butoxycarbonyl)-N-(4-trifluoromethylbenzyl)aminomethyl]-4-methoxydihydrocinnamic acid (34.60 g, 71 mmol) and pyridine (6.9 mL, 85 mmol) in dry dichloromethane (200 mL) under Argon was added cyanuric fluoride (4.4 mL, 48 mmol) via syringe. The reaction was stirred for 4 h at RT. The resulting thick suspension was filtered through a pad of celite® and rinsed with a small volume of dry dichloromethane (50 mL). The clear filtrate was poured into a separatory funnel and washed with ice-cold water (500 mL). Drying (MgSO₄) and concentration left the crude acid fluoride (34.70 g, 100%) which was used without further purification.

To a stirred solution of (R)-4-benzyl-2-oxazolidinone (13.8 g, 78 mmol) in dry THF (300 mL) under argon at −78° C. was added via syringe a solution of n-BuLi in hexanes (2.5 M, 30 mL, 75 mmol). The reaction was stirred at −78° C. for 15 min, then a solution of the above acid fluoride (34.70 g, 71 mmol) in dry THF (100 nL) was added via syringe. The reaction was stirred for 1 h at −78° C. then was quenched with saturated NH₄Cl and extracted with ethyl acetate (2×200 mL). The combined organic layers were washed with brine (400 mL), dried (MgSO₄), and concentrated to dryness. Purification by flash chromatography on silica gel (20% ethyl acetate/hexanes) gave the title compound (40.34 g, 90%) as a thick clear oil: TLC (20% EtOAc/hexane) R_{f} 0.21; ¹H NMR (400 MHz, CDCl₃) δ 7.58 (d, J=8.1 Hz, 2H), 7.33-7.26 (m, 5H), 7.16 (m, 3H), 6.77 (dd, 1H), 6.67 (d, J=2.5 Hz, 1H), 4.62 (m, 1H), 4.60-4.40 (m, 4H), 4.16 (m, 2H), 3.76 (s, 3H), 3.27 (dd, 1H), 3.21-3.10 (m, 2H), 2.88 (br s, 2H), 2.72 (dd, 1H), 1.48 (s, 9H).

h) (R)-4-Benzyl-2-oxazolidinonyl 3-[2-[N-(tert-butoxycarbonyl)-N-(4-trifluoromethylbenzyl)aminomethyl]-4-methoxyphenyl]-2(S)-methoxycarbonyimethyl-propionamide

To a stirred solution of (R)-4-benzyl-2-oxazolidinonyl 2-[N-(tert-butoxycarbonyl)-N-(4-trifluormethylbenzyl)aminomethyl]-4-methoxydihydrocinnamide (40.30 g, 64 mmol) in dry THF (300 mL) at −78° C. was added a solution of lithium bis(trimethylsilyl)amide (70 mL, 1 M in THF, 70 mmol) via syringe. After 30 min, methyl bromoacetate (30 mL, 317 mmol) was added via syringe. After another 30 min at −78° C. the reaction was allowed to warm to −20° C. and stirred for an additional 6 h. The reaction was quenched with saturated NH₄Cl (400 mL) and extracted with ethyl acetate (2×200 mL). The combined organic layers were washed with brine (300 mL), dried (MgSO₄), and concentrated to dryness. Purification by flash chromatography on silica gel (20% ethyl acetate/hexanes) gave the title compound (38.62 g, 86%) as a white solid: HPLC (Altex Ultrasphere™-Si 5μ, 20% EtOAc/hexane) showed approximately 20% unalkylated starting material was still present. HPLC of the crude reaction mixture gave a de of 90% for the reaction; ¹H NMR (400 MHz, CDCl₃) δ 7.57 (d, J=8.1 Hz, 2H), 7.40-7.11 (m, 8H), 6.71 (dd, 1H), 6.63 (d, J=2.7 Hz, 1H), 4.57-4.34 (m, 6H), 4.03 (d, J=8.6 Hz, 1H), 3.85 (t, 1H), 3.72 (s, 3H), 3.61 (s, 3H), 3.28 (dd, 1H), 2.90 (dd, 1H), 2.86-2.71 (m, 2H), 2.70 (dd, 1H), 2.44 (m, 1H), 1.48 and 1.46 (2s, 9H).

i) Methyl (S)-8-methoxy-3-oxo-2-(4-trifluoromethylbenzyl)-2, 3, 4, 5-tetrahydro-1H-2-benzazepine-4-acetate

To a stirred solution of (R)-4-benzyl-2-oxazolidinonyl 3-[2-[N-(tert-butoxycarbonyl)-N-(4-trifluoromethylbenzyl)aminomethyl]-4-methoxyphenyl]-2(S)-methoxycarbonylmethyl-propionamide (38.0 g, 54 mmol) in THF (300 mL) and water (100 mL) was added dropwise at 0° C. over 30 min a solution of 30% H₂O₂ (18.9 mL) and LiOH.H₂O (2.3 g, 55 mmol) in water (62 mL). The cloudy solution was stirred for an additional 1 h at 0° C. The resulting homogeneous solution was treated slowly with a solution of sodium sulfite (34.3 g, 272 mmol) in water (175 mL) at 0° C., then was acidified with an ice-cold solution of concentrated HCl (35 mL) in water (150 mL). The reaction was extracted with ethyl acetate (2×200 mL), and the combined organic layers were washed with brine (400 mL), dried (MgSO₄) and concentrated to dryness. The resulting residue was treated with 4.0 M HCl in dioxane (400 mL) with stirring at RT (slow gas evolution was observed). After 1 h, the reaction was concentrated and reconcentrated from 1:1 CHCl₃/toluene (2 x), then the residue (37.65 g) was taken up in dry DMF (400 mL). To this solution with stirring under argon at 0° C. in a Dewar flask were added triethylamine (15.3 mL, 109 mmol) and NaHCO₃ (22.9 g, 273 mmol), followed by diphenylphosphoryl azide (13 mL, 60 mmol). After stirring for 24 h at 0° C. the reaction was concentrated to dryness. The residue was taken up in ethyl acetate (400 mL), and washed sequentially with water (300 mL) and brine (300 mL). Drying (MgSO₄), concentration, and flash chromatography on silica gel (35% ethyl acetate/hexanes) gave the title compound (16.87 g, 74%) as a clear thick oil: TLC (40% EtOAc/hexane) R_{f} 0.50; MS (ES) m/e 422.3 (M+H)⁺; ¹H NMR (400 MHz, CDCl₃) δ 7.52 (d, J=8.1, 2H), 7.29 (d, J=8.1 Hz, 2H), 7.02 (d, J=8.5 Hz, 1H), 7.75 (dd, 1H), 6.36 (d, J=2.7 Hz, 1H), 5.18 (d, J=16.5 Hz, 1H), 4.96 (d, J=15.4 Hz, 1H), 4.48 (d, J=15.4 Hz, 1H), 3.87 (m, 1H), 3.74 (d, J=16.5 Hz, 1H), 3.73 (s, 3H), 3.71 (s, 3H), 3.08 (dd, 1H), 3.02 (dd, 1H), 2.95 (dd, 1H), 2.48 (dd, 1H).

j) Methyl (S)-8-hydroxy-3-oxo-2-(4-trifluoromethylbenzyl)-2, 3, 4, 5-tetrahydro- 1H-2-benzazepine-4-acetate

A solution of boron tribromide in CH₂Cl₂ (1.0 M, 160 mL, 160 mmol) was added dropwise over 30 min to a solution of methyl (S)-8-methoxy-3-oxo-2-(⁴-trifluoromethylbenzyl)-2, 3, 4, 5-tetrahydro-1H-2-benzazepine4acetate (16.67 g, 39.6 mmol) in anhydrous CH₂Cl₂ (150 mL) at −20° C. under argon. After an additional 1.5 hr at −15 to −20° C., the reaction was recooled to −20° C. and quenched by careful dropwise addition of MeOH (160 mL). The reaction was stirred at −10 to 0° C. for 1 hr, then was concentrated on the rotavap. The residue was reconcentrated from MeOH (2 x). Purification by flash chromatography on silica gel (50 to 100% ethyl acetate/hexanes) gave the title compound (14.87 g, 92%) as a white solid: [α]_{D}−81.8° (c, 1.0, MeOH); TLC (silica, 50% EtOAc/hexane) R_{f}0.54; MS (ES) m/e 408.2 (M+H)⁺; ¹H NMR (400, CDCl₃+2% DMSO-d₆) δ 7.53 (d, J=8.1 Hz, 2H), 7.31 (d, J=8.1 Hz, 2H), 6.93 (d, J=8.4 Hz, 1H), 6.70 (dd, 1H), 6.41 (d, J=2.3 Hz, 1H), 5.16 (d, J=16.4 Hz, 1H), 5.01 (d, J=15.6 Hz, 1H), 4.39 (d, J=15.6 Hz, 1H), 3.84 (m, 1H), 3.73 (d, J=16.4 Hz, 1H), 3.71 (s, 3H), 3.01 (dd, 1H), 2.98 (m, 1H), 2.90 (dd, 1H), 2.47 (dd, 1H).

### Preparation 7

### Preparation of 2-[(3-hydroxy-1-propyl)amino]-4-methylpyridine-N-oxide

a) 2-[(3-hydroxy-1-propyl)amino]-4-methylpyridine-N-oxide

A mixture of 2-chloro-4-methylpyridine-N-oxide (12.1 g, 0.068 mole) (Brown, E. V. J. Amer. Chem. Soc. 1957, 79, 3565), 3-amino-1-propanol (10.33 mL,0.14 mole), NaHCO₃ (28 g, 0.34 mole), and tert-amyl alcohol (70 mL) was heated to reflux. After 16 hr, the reaction was cooled, diluted with CH₂Cl₂ (300 mL), and suction filtered to remove insoluble materials. The filtrate was concentrated and reconcentrated from toluene to leave a yellow oil. Recrystallization from CH₂Cl₂/Et₂O gave the title compound (10.87 g, 88%) as a yellow solid: TLC (15% MeOH/CH₂Cl) R_{f}0.44; ¹H NMR (400, CDCl₃) δ 7.92 (d, J=6.7, 1 H), 7.28 (br t, 1 H), 6.43 (s, 1 H), 6.33 (dd, J=6.6, 2.1 Hz, 1H), 3.73 (t, J=5.7 Hz, 2 H), 3.47 (q, H=6.3 Hz, 2 H), 2.29 (s, 3 H), 1.82-1.88 (m, 2 H); MS (ES) m/e 183 (M+H)⁺.

### Preparation 8

### Preparation of (S)-8-[3-(4-methylpyridin-2-ylamino)-1-propyloxy]-3-oxo-2-[4-(trifluoromethyl)benzyl]-2,3,4,5-tetrahydro-1H-2-benzazepineacetic acid

a) Methyl (S)-8-[3-(4-methyl-1-oxopyridin-2-ylamino)-1-propyloxy]-3-oxo-2-[4-(trifluoromethyl)benzyl]-2,3,4,5-tetrahydro-1H-2-benzazepine-4-acetate

To methyl (S)-8-hydroxy-3-oxo-2-[4-(trifluoromethyl)benzyl]-2, 3, 4, 5-tetrahydro-1H-2-benzazepine-4-acetate (0.42 g, 1.03 mmol) and Ph₃P (0.41 g, 1.56 mmol) in CH₂Cl₂ (6 mL) at 0° C. was added dropwise a solution of 2-[(3-hydroxy-1-propyl)amino]-4-methylpyridine-N-oxide (0.28 g, 1.54 mmol) and diethyl azodicarboxylate (0.24 mL, 1.52 mL). When the addition was complete, the ice bath was removed and the reaction was stirred at RT. After 20 h, the solvent was removed and the product was isolated by flash chromatography (100% CHCl₃ to 10% MeOH/CHCl₃, silica gel) to give the title compound (0.57 g) as a clear oil. MS (ES) m/e 572.2 (M+H)⁺.

b) Methyl (S)-8-[3-(4methylpyridin-2-ylamino)-1-propyloxy]-3-oxo-2-[4-(trifluoromethyl)benzyl]-2,3,4,5-tetrahydro-1H-2-benzazepine-4-acetate

To methyl (S)-8-[3-(4-methyl-1-oxo-pyridin-2-ylamino)-1-propyloxy]-3-oxo-2-[4-(trifluoromethyl)benzyl]-2,3,4,5-tetrahydro-1H-2-benzazepine-4-acetate (0.57 g, 1.00 mmol) and cyclohexene (1.00 mL, 9.87 mmol) in MeOH (10 mL) was added 10% Pd/C (0.11 g). The reaction was heated to reflux for 20 h. After cooling the reaction to RT, the catalyst was removed by filtration and the solvent was removed under vacuum to give a white foam (0.49 g). Radial chromatography (5% MeOH/CHCl₃, silica gel, 6 mm plate) gave the title compound (0.42 g) as a white foam. MS (ES) m/e 556.1 (M+H)⁺.

c) (S)-8-[3-(4-Methylpyridin-2-ylamino)-1-propyloxy]-3-oxo-2-[4-(trifluoromethyl)benzyl]-2,3,4 5-tetrahydro-1H-2-benzazepine-4-acetic acid

To methyl (S)-8-[3-(4-methylpyridin-2-ylamino)-1-propyloxy]-3-oxo-2-[4-(trifluoromethyl)benzyl]-2,3,4,5-tetrahydro-1H-2-benzazepine-4-acetate (0.42 g, 0.75 mmol) in EtOH (2 mL) was added 1N NaOH (1.50 mL, 1.50 mmol). After stirring at RT for 3 h, the bulk of the solvent was removed under vacuum. The residue was made acidic (pH=3) with 1N HCl, frozen and lyophilized to dryness. Water was added to the residue and neutralized to pH=7 with sat NaHCO₃. The aqueous layer was extracted with CHCl₃, the combined organic extracts were dried over Na₂SO₄ and concentrated to give a white foam (0.47 g). Radial chromatography (10% MeOH/CHCl₃, silica gel, 6 mm plate) gave the title compound (0.30 g) as a white solid. MS (ES) m/e 542.2 (M+H)⁺. Anal. Calcd for C₂₉H₃₀F₃N₃O₄.3.5 H₂O: C, 57.61; H, 6.17; N, 6.95. Found: C, 57.6; H, 5.30; N, 6.38.

### Example 2

### Parenteral Dosage Unit Composition

A preparation which contains 20 mg of the compound of Example 1 as a sterile dry powder is prepared as follows: 20 mg of the compound is dissolved in 15 mL of distilled water. The solution is filtered under sterile conditions into a 25 mL multi-dose ampoule and lyophilized. The powder is reconstituted by addition of 20 mL of 5% dextrose in water (D5W) for intravenous or intramuscular injection. The dosage is thereby determined by the injection volume. Subsequent dilution may be made by addition of a metered volume of this dosage unit to another volume of D5W for injection, or a metered dose may be added to another mechanism for dispensing the drug, as in a bottle or bag for IV drip infusion or other injection-infusion system.

### Example 3

### Oral Dosage Unit Composition

A capsule for oral administration is prepared by mixing and milling 50 mg of the compound of Example 1 with 75 mg of lactose and 5 mg of magnesium stearate. The resulting powder is screened and filled into a hard gelatin capsule.

### Example 4

### Oral Dosage Unit Composition

A tablet for oral administration is prepared by mixing and granulating 20 mg of sucrose, 150 mg of calcium sulfate dihydrate and 50 mg of the compound of Example 1 with a 10% gelatin solution. The wet granules are screened, dried, mixed with 10 mg starch, 5 mg talc and 3 mg stearic acid; and compressed into a tablet.

The above description fully discloses how to make and use the present invention. However, the present invention is not limited to the particular embodiments described hereinabove, but includes all modifications thereof within the scope of the following claims. The various references to journals, patents and other publications which are cited herein comprises the state of the art and are incorporated herein by reference as though fully set forth.

What is claimed is:

1. A compound according to formula (I): 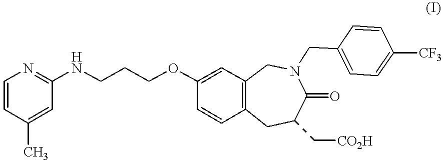
or a pharmaceutically acceptable salt thereof.

2. A pharmaceutical composition which comprises a compound according to claim 1 and a pharmaceutically acceptable carrier.

3. A pharmaceutical composition which comprises a compound according to claim 1, an antineoplastic agent and a pharmaceutically acceptable carrier.

4. The pharmaceutical composition according to claim 3 wherein the antineoplastic agent is topotecan.

5. The pharmaceutical composition according to claim 3 wherein the antineoplastic agent is cisplatin.

6. A method of treating a disease state in which antagonism of the α_{V}β₃ receptor is indicated which comprises administering to a subject in need thereof a compound according to claim 1.

7. A method of treating a disease state in which antagonism of the α_{V}β₅ receptor is indicated which comprises administering to a subject in need thereof a compound according to claim 1.

8. A method of treating osteoporosis which comprises administering to a subject in need thereof a compound according to claim 1.

9. A method for inhibiting angiogenesis which comprises administering to a subject in need thereof a compound according to claim 1.

10. A method for inhibiting tumor growth or tumor metastasis which comprises administering to a subject in need thereof a compound according to claim 1.

11. A method of treating atherosclerosis or restenosis which comprises administering to a subject in need thereof a compound according to claim 1.

12. A method of treating inflammation which comprises administering to a subject in need thereof a compound according to claim 1.

13. A method of inhibiting tumor growth which comprises administering stepwise or in physical combination a compound according to claim 1 and an antineoplastic agent.

14. The method according to claim 13 wherein the antineoplastic agent is topotecan.

15. The method according to claim 13 wherein the antineoplastic agent is cisplatin.

16. A compound according to formula (II): 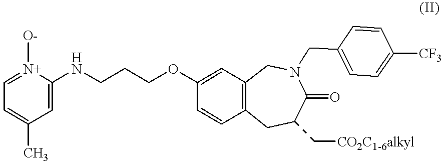
or a pharmaceutically acceptable salt thereof.

17. A compound according to formula (III): 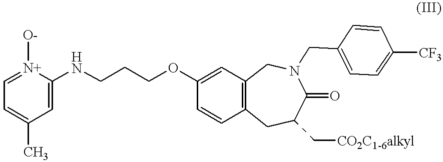
or a pharmaceutically acceptable salt thereof.

18. A compound according to claim 1 for use as a medicament.

19. The use of a compound of claim 1 in the manufacture of a medicament for the treatment of diseases in which antagonism of the (α_{V}β₃ receptor is indicated.

20. The use of a compound of claim 1 in the manufacture of a medicament for the treatment of diseases in which antagonism of the α_{V}β₅ receptor is indicated.

21. The use of a compound of claim 1 in the manufacture of a medicament for the treatment of osteoporosis.

22. The use of a compound of claim 1 in the manufacture of a medicament for the inhibition of angiogenesis.

23. The use of a compound of claim 1 in the manufacture of a medicament for the inhibition of tumor growth or tumor metastasis.

24. The use of a compound of claim 1 in the manufacture of a medicament for the treatment of atherosclerosis or restenosis.

25. The use of a compound of claim 1 in the manufacture of a medicament for the treatment of inflammation.

26. The use of a compound of claim 1 and an antineoplastic agent in the manufacture of a medicament for the inhibition of tumor growth in physical combination or for stepwise administration.

27. The use according to claim 26 wherein the antineoplastic agent is topotecan.

28. The use according to claim 26 wherein the antineoplastic agent is cisplatin.

29. The use of a compound of claim 1 and an inhibitor of bone resorption in the manufacture of a medicament for the treatment of osteoporosis in physical combination or for stepwise administration.
